(19) United States
(12) Patent Application Publication
(10) Pub. No.: US 2024/0164856 A1
(43) Pub. Date: May 23, 2024

(54) DETECTION IN A SURGICAL SYSTEM

(71) Applicant: Verb Surgical Inc., Santa Clara, CA (US)

(72) Inventors: Rami Azmi Nimer Abukhalil, West Chester, OH (US); Kenneth Fernandez Prada, Santa Clara, CA (US); Amer G. Ghanem, West Chester, OH (US); Mary Lynn Dwyer-Gaddis, Newport Beach, CA (US)

(21) Appl. No.: 17/991,566

(22) Filed: Nov. 21, 2022

(51) Int. Cl. A61B 34/32; A61B 1/267; A61B 34/20; G06N 20/00

(52) U.S. Cl. CPC A61B 34/32; A61B 1/2676; A61B 34/20; G06N 20/00; A61B 2034/2065

(57) ABSTRACT

For intraoperative guidance to an object, a machine-learned model is used to predict the intra-operative location of an object identified in pre-operative planning. The predicted location is used by the surgeon or controller during the operation, such as during a bronchoscopy.

### FIELD

Embodiments relate to lung mass detection in a surgical system.

### BACKGROUND

Minimally invasive surgery (MIS), such as laparoscopic surgery, involves techniques intended to reduce tissue damage during a surgical procedure. MIS may be performed with robotic systems that include one or more robotic arms for manipulating surgical tools based on commands from a remote operator or with other surgical systems where a user guides a tool within the patient.

MIS may be used in surgical procedures on the lungs or other organ of the patient, such as for an operation, biopsy, or viewing a lung module, mass or lymph node. Detection of masses, nodules, and intra-segmental lymph nodes intra-operatively is sometimes challenging for surgeons. For example, thoracic surgeons have difficulty detecting lung masses due to deflation of the lung during the procedure. In MIS, the lung is deflated or deformed relative to pre-operative images. Masses previously located by a computed tomography (CT) pre-operative scan may not be detected easily intra-operatively due to lungs changes from surgery. The difficulty is even greater for deep (non-superficial) lung objects.

### SUMMARY

By way of introduction, the preferred embodiments described below include methods, systems, instructions, and computer readable media for intraoperative guidance to an object in a patient, such as a lung object. A machine-learned model is used to predict the intra-operative location of an object identified in pre-operative planning. The predicted location is used by the surgeon or controller during the operation, such as during a bronchoscopy.

In a first aspect, a method is provided for intraoperative guidance to an object for a surgical system. A location of the object is indicated during surgery. A machine-learned model indicates the location. The machine-learned model outputs the location in response to input of information to the machine-learned model. The surgical system is guided to the location based on the indication.

In one embodiment, the guidance is by overlaying the location on an image generated during the surgery. In another embodiment, the guidance is also based on a detected location of a surgical instrument relative to the location of the object. For example, a controller of the surgical system guides using the detected location of the surgical instrument and the indicated location of the object.

According to one embodiment, the machine-learned model is a convolutional neural network, a U-Net, or an encoder-decoder. According to other embodiments, various information may be input to the machine-learned model. For example, the information is a pre-operative image of a patient and a real-time image of the patient in the surgery, such as a pre-operative computed tomography image and a real-time image computed tomography or x-ray image. As another example, the information includes a pre-operative image of a lung and where the location is on the lung with the lung deflated relative to the lung in the pre-operative image. The location is a predicted intra-operative location. In yet another example, the information includes patient clinical data and/or breathing cycle data.

The object is a mass, a nodule, or an intra-segmental lymph node. Other objects may be indicated, such as landmarks of the lung.

As another embodiment, a bronchoscope of the surgical system is guided in the patient. As yet another embodiment, a surgical robotic arm is controlled to guide a surgical instrument to the location.

In a second aspect, a medical system is provided for intra-operative prediction of a location of an object in a deflated lung. A memory is configured to store a machine-trained model. The machine-trained model was trained from training images including pre-operative images of sample objects and ground truth including positions of the sample objects during surgery. A processor is configured to predict the location of the object for a patient during the surgery. The location is predicted by the machine-trained model in response to input of a pre-operative image of the patient and a position of the object in the pre-operative image to the machine-trained model. An output interface is configured to output the location of the object as predicated.

In one embodiment, the output interface connects to a display. The location is shown on the display.

In another embodiment, the output interface is configured to output the location to a bronchoscope navigation system or a surgical robotic system.

As another embodiment, the input to the machine-trained model is the pre-operative image of the patient, the position of the object in the pre-operative image, and patient clinical data.

For another embodiment, the pre-operative image of the patient is a computed tomography image, and the input further includes a real-time x-ray or computed tomography image of the patient during the surgery.

In a third aspect, a method is provided for machine training for intra-operative object predication. Pre-operative images of lungs with objects and intra-operative locations of the objects with the lungs deflated relative to the lungs of the pre-operative images are obtained. A machine learning model is machine trained to output the locations of the intra-operative objects in response to input of the pre-operative images. The machine learning model as trained is stored.

According to one embodiment, the machine training includes machine training for the output in response to input of the pre-operative images, pre-operative locations of the objects, intra-operative images of the lungs, and clinical data. In other embodiments, the pre-operative images and the intra-operative images are obtained as computed tomography images.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The embodiments of the invention are illustrated by way of example and not by way of limitation in the figures of the accompanying drawings in which like references indicate similar elements. It should be noted that references to “an” or “one” embodiment of the invention in this disclosure are not necessarily to the same embodiment, and they mean at least one. Also, in the interest of conciseness and reducing the total number of figures, a given figure may be used to illustrate the features of more than one embodiment of the invention, and not all elements in the figure may be required for a given embodiment.

FIG. 1 illustrates an embodiment of a cart-based robotic system arranged for diagnostic and/or therapeutic use;

FIG. 2 illustrates an embodiment of a table-based robotic system configured for a procedure;

FIG. 3 is a block diagram of another embodiment of a surgical system using a machine-learned model to predict lung object position;

FIG. 4 illustrates both training and application of a machine-learned model for lung object prediction;

FIG. 5 is a flow chart diagram of one embodiment of a method for machine learning to predict object location intra-operatively;

FIG. 6 is a flow chart diagram of one embodiment of a method for prediction of a position of an object during surgery using a machine-learned model; and

FIGS. 7-9 illustrate example guidance based on artificial intelligence-based object location prediction.

### DETAILED DESCRIPTION

Artificial intelligence detects one or more objects in a patient, such as a mass, lesion, lymph node, landmark, screw, stent, or instrument. The example used herein is a lung object, such as a mass. Other objects inside the patient than a lung object may be detected.

Artificial intelligence detects one or more lung objects (e.g., lung nodule, mass, landmark, and/or lymph node) with non-invasive procedures. The machine learning model is trained to predict the place of the object on or in the deflated lung during surgery. The training data may include pre-operative CT scans with object locations and corresponding intra-operative mass locations. Once trained, the predicted place is provided to the surgeon and/or surgical system in real time and/or during the procedure. This machine-learned model is useful for surgeons by providing more accurate guidance and resolves the issue where the lung deflates (shrinks) intra-operatively, leading to anatomical changes that confuse a surgeon.

The example surgical procedure used herein is a bronchoscopy, such as for diagnosis. A bronchoscope is inserted within the lungs of the patient to better view a lung object for diagnosis. Other surgical procedures on the lungs may be provided, such as for biopsy or treatment (e.g., removal or application of pharmaceutical or energy).

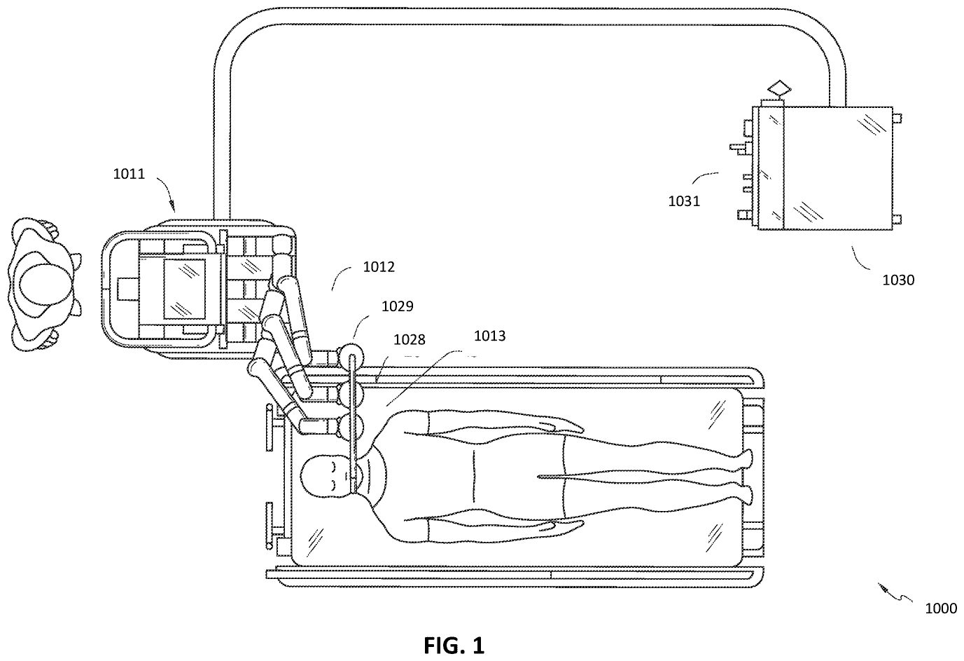

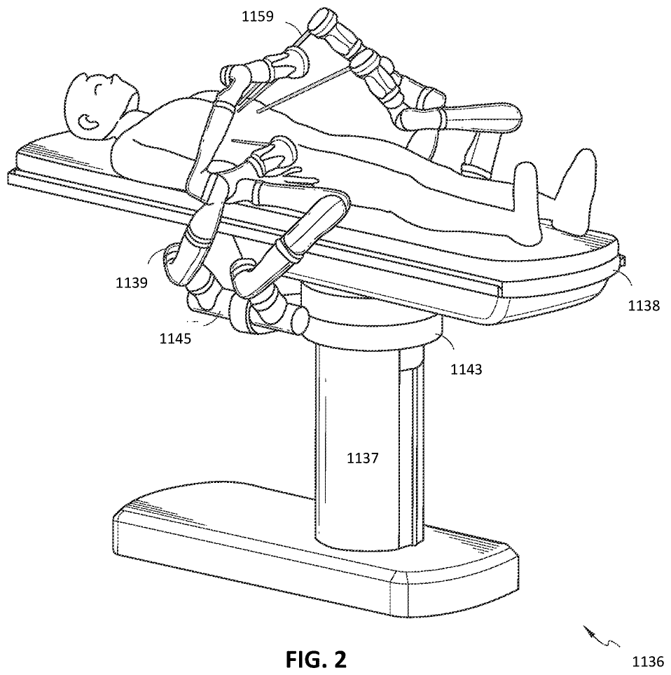

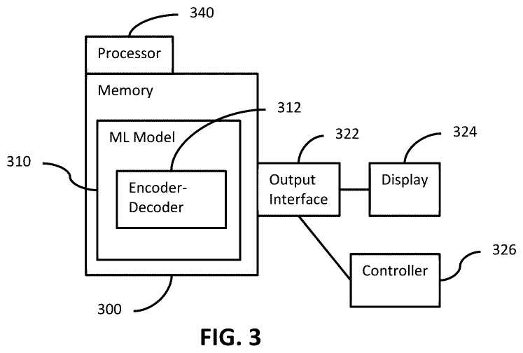

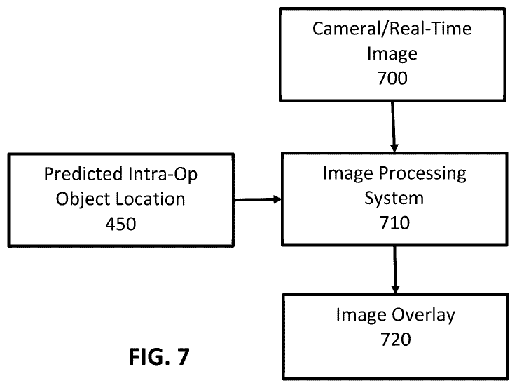

The discussion below first introduces an example robotic surgery system (see FIGS. 1 and 2). Other surgical systems, such as under manual control of a surgeon, may be used. FIGS. 3-6 are directed to creation (training) and use of a machine-learned model in prediction of intra-operative lung object position. FIGS. 7-9 show embodiments for guidance based on artificial intelligence prediction of intra-operative position of a lung object.

The robotic surgical system may have any arrangement, such as one or more robotic arms. One or more surgical instruments may be used, such as graspers, clamps, endoscope, and/or scalpel instruments. FIGS. 1 and 2 show an example robotic surgery system. Other surgical instruments, robotic arms, and/or robotic surgery systems may be used.

FIG. 1 shows an embodiment of a surgical robotic system. The robotically-enabled medical system may be configured in a variety of ways depending on the particular procedure. In FIG. 1, the surgical robotic system is a cart-based robotically-enabled system 1000 arranged for a diagnostic and/or therapeutic surgery, such as bronchoscopy. The system 1000 may include a cart 1011 having one or more robotic arms 1012 to deliver a medical instrument, such as a steerable endoscope 1013 and/or therapeutic tools. The cart 1011 may be positioned proximate to the patient's upper torso to provide access to an access point. Similarly, the robotic arms 1012 may be actuated to position the instruments. The arrangement in FIG. 1 with the arms 1012 repositioned may be utilized when performing other surgeries (e.g., a gastro-intestinal (GI) procedure with a gastroscope, RYGB, or a specialized endoscope for GI procedures).

Once the cart 1011 is properly positioned, the robotic arms 1012 may insert the steerable endoscope 1013 into the patient robotically, manually, or a combination thereof. The steerable endoscope 1013 may include at least two telescoping parts, such as an inner leader portion and an outer sheath portion, each portion coupled to a separate instrument driver from the set of instrument drivers 1028, each instrument driver coupled to the distal end of an individual robotic arm 1012. This linear arrangement of the instrument drivers 1028, which facilitates coaxially aligning the leader portion with the sheath portion, creates a “virtual rail” 1029 that may be repositioned in space by manipulating the one or more robotic arms 1012 into different angles and/or positions. The virtual rails described herein are not any physical structure of the system but an arrangement of other structures. Translation of the instrument drivers 1028 along the virtual rail 1029 telescopes the inner leader portion relative to the outer sheath portion or advances or retracts the endoscope 1013 from the patient. The angle of the virtual rail 1029 may be adjusted, translated, and pivoted based on clinical application or physician preference. For example, in bronchoscopy, the angle and position of the virtual rail 1029 as shown represents a compromise between providing physician access to the endoscope 1013 while minimizing friction that results from bending the endoscope 1013 into the patient's mouth. Similarly, for RYGB, the endoscope is inserted through a port in the patient, so the angle and position of the virtual rail 1029 is oriented about that access point. The virtual rail may not be used for some procedures, such as RYGB.

The endoscope 1013 may be directed within the patient after insertion using precise commands from the robotic system until reaching the target destination or operative site. To enhance navigation and/or reach the desired target, the endoscope 1013 may be manipulated to telescopically extend the inner leader portion from the outer sheath portion to obtain enhanced articulation and greater bend radius. The use of separate instrument drivers 1028 also allows the leader portion and sheath portion to be driven independently of each other.

The system 1000 may also include a movable tower 1030, which may be connected via support cables to the cart 1011 to provide support for controls, electronics, fluidics, optics, sensors, and/or power to the cart 1011. Placing such functionality in the tower 1030 allows for a smaller form factor cart 1011 that may be more easily adjusted and/or re-positioned by an operating physician and his/her staff. Additionally, the division of functionality between the cart/table and the support tower 1030 reduces operating room clutter and facilitates improving clinical workflow. While the cart 11 may be positioned close to the patient, the tower 1030 may be stowed in a remote location to stay out of the way during a procedure.

In support of the robotic systems described above, the tower 1030 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 1030 or the cart 1011, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 1030 may also include a pump, flow meter, valve control, and/or fluid access to provide controlled irrigation and aspiration capabilities to the system that may be deployed through the endoscope 1013. The tower 30 may include a voltage and surge protector designed to provide filtered and protected electrical power to the cart 11, thereby avoiding placement of a power transformer and other auxiliary power components in the cart 1011, resulting in a smaller, more moveable cart 1011. The tower 1030 may also include support equipment for the sensors deployed throughout the robotic system 1000. Similarly, the tower 1030 may also include an electronic subsystem for receiving and processing signals received from deployed electromagnetic (EM) sensors. The tower 1030 may also be used to house and position an EM field generator for detection by EM sensors in or on the medical instrument. Similarly, the tower 1030 may include a processor connected to a breathing sensor for monitoring the breathing cycle of the patient.

The tower 1030 may also include a console 1031 in addition to other consoles available in the rest of the system, e.g., console mounted on top of the cart. The console 1031 may include a user interface and a display screen, such as a touchscreen, for the physician operator. Consoles in the system 1000 are generally designed to provide both robotic controls as well as preoperative and real-time information of the procedure, such as navigational and localization information of the endoscope 1013. When the console 1031 is not the only console available to the physician, it may be used by a second operator, such as a nurse, to monitor the health or vitals of the patient and the operation of the system 1000, as well as to provide procedure-specific data, such as navigational and localization information. In other embodiments, the console 1031 is housed in a body that is separate from the tower 1030.

Embodiments of the robotically-enabled medical system may also incorporate the patient's table. Incorporation of the table reduces the amount of capital equipment within the operating room by removing the cart, which allows greater access to the patient. FIG. 2 illustrates an embodiment of such a robotically-enabled system. The system 1136 includes a support structure or column 1137 for supporting platform 1138 (shown as a “table” or “bed”) over the floor. Much like in the cart-based systems, the end effectors of the robotic arms 1139 of the system 1136 comprise instrument drivers that are designed to manipulate an elongated medical instrument, such as an endoscope, grasper, and/or scalpel.

The column 1137 may include one or more carriages 1143 shown as ring-shaped in the system 1136, from which the one or more robotic arms 1139 may be based. The carriages 1143 may translate along a vertical column interface that runs the length of the column 1137 to provide different vantage points from which the robotic arms 1139 may be positioned to reach the patient. The carriage(s) 1143 may rotate around the column 1137 using a mechanical motor positioned within the column 1137 to allow the robotic arms 1139 to have access to multiples sides of the table 1138, such as, for example, both sides of the patient. In embodiments with multiple carriages 1143, the carriages 1143 may be individually positioned on the column 1137 and may translate and/or rotate independently of the other carriages. While the carriages 1143 need not surround the column 1137 or even be circular, the ring-shape as shown facilitates rotation of the carriages 1143 around the column 1137 while maintaining structural balance. Rotation and translation of the carriages 1143 allows the system 1136 to align the medical instruments into different access points on the patient. In other embodiments (not shown), the system 1136 can include a patient table or bed with adjustable arm supports in the form of bars or rails extending alongside it. One or more robotic arms 1139 (e.g., via a shoulder with an elbow joint) can be attached to the adjustable arm supports, which can be vertically adjusted. By providing vertical adjustment, the robotic arms 1139 are advantageously capable of being stowed compactly beneath the patient table or bed, and subsequently raised during a procedure.

The robotic arms 1139 may be mounted on the carriages 1143 through a set of arm mounts 1145 including a series of joints that may individually rotate and/or telescopically extend to provide additional configurability to the robotic arms 1139. Additionally, the arm mounts 1145 may be positioned on the carriages 1143 such that, when the carriages 1143 are appropriately rotated, the arm mounts 1145 may be positioned on either the same side of the table 1138, on opposite sides of the table 1138 (as shown in FIG. 11), or on adjacent sides of the table 1138 (not shown).

The column 1137 structurally provides support for the table 1138, and a path for vertical translation of the carriages 1143. Internally, the column 1137 may be equipped with lead screws for guiding vertical translation of the carriages, and motors to mechanize the translation of the carriages 1143 based on the lead screws. The column 1137 may also convey power and control signals to the carriages 1143 and the robotic arms 1139 mounted thereon.

In one embodiment, the robotic surgical system of FIG. 2 is used for RYGB. In a laparoscopic procedure, through small incision(s) in the patient's abdominal wall, minimally invasive instruments may be inserted into the patient's anatomy. In some embodiments, the minimally invasive instruments include an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments include a scope, such as a laparoscope for a viewing task, not necessarily surgical in the sense of operating directly on tissue, one or more graspers for manipulating intestine, and a scalpel for creating a bypass. FIG. 2 illustrates an embodiment of a robotically-enabled, table-based system configured for a laparoscopic procedure, such as RYGB. As shown in FIG. 2, the carriages 1143 of the system 1136 may be rotated and vertically adjusted to position pairs of the robotic arms 1139 on opposite sides of the table 1138, such that instruments 1159 may be positioned using the arm mounts 1145 to be passed through minimal incisions (ports) on both sides of the patient to reach his/her abdominal cavity. RYGB may then be performed using one or more drivers with the surgical instruments or tools.

In other embodiments, the robotic arms 1139 and surgical instruments 1159 are positioned for bronchoscopy. For example, the system 1136 positions to form the virtual rail 1029 of FIG. 1. Alternatively, one of the robotic arms 1139 and corresponding bronchoscope as the surgical instrument 1159 are positioned for insertion of the bronchoscope through the mouth or nose of the patient for guidance to and within the lungs. In yet other embodiments, the surgical system does not include robotics. Instead, the surgeon moves the instrument 1159 (e.g., bronchoscope) based on position assistance from artificial intelligence.

FIG. 3 shows one embodiment of a surgical system for prediction of lung object location, such as for intra-operative prediction of a location of a lung object in a deflated lung. The system is a workstation, computer, controller, server, tablet, or mobile device. In one embodiment, the system is part of the surgical robotic system, such as being in the tower 1030 or cart 1011. In another embodiment, the system communicates wirelessly and/or wired (e.g., over a computer network) with the robotic system 1000, 1136.

Via communications and/or physical connection, the system of FIG. 3 connects with one or more of the robotic arms, such as connecting with the surgical robot or system 1000, 1136. Interaction with the arms 1139 of FIG. 2 will be used in examples below, but interaction with other robotic arms (e.g., arms 1012 of FIG. 1) may be used in other examples. The robotic arm(s) 1139 are configured to hold and operate surgical tool(s) 1159. In alternative embodiments, a surgical system is provided without robotic arms, such as for guiding a surgeon manually or directly guiding a bronchoscope.

The surgical system includes a processor 340, a memory 300, an output interface 322, a controller 326, and a display 342. Additional, different, or fewer devices may be provided, such as not providing the display 324 and/or not providing the controller 326.

The memory 300 is a random-access memory, hard drive, read only memory, complementary metal-oxide semiconductor, flash, or other memory hardware. The memory 300 is configured by the processor 340 or another processor to store data. For example, the memory 300 is configured to store the machine-learned (ML) model 310. During training, machine learning is used to train based on training data, such as pre-operative images of sample objects and ground truth of positions of the sample objects during surgery. The training data may be stored in the memory 300. Once machine learning is performed, the model 310 as trained with the training data is stored in the same or different memory 300.

The memory 300 may be a non-transitory memory configured to store instructions executable by the processor 340. Alternatively, a separate memory is used for the processor instructions.

The processor 340 is a general processor, application specific integrated circuit, field programmable gate array, digital signal processor, controller, artificial intelligence processor, tensor processor, graphics processing unit, digital circuit, analog circuit, combinations thereof, and/or other now known or later developed processor for predicting a location or locations of one or more lung objects for a patient during surgery. The processor 340 is configured by software, hardware, and/or firmware to apply the artificial intelligence and/or determine the location for each of one or more lung objects.

The processor 340 uses the ML model 310 to predict the location of the lung object for a patient during the surgery. The prediction occurs while the surgery is being performed (e.g., while the bronchoscope is within the patient). The predication may be in real-time, such as within a one or two seconds of capturing the input to the ML model 310. In alternative embodiments, the prediction occurs prior to surgery.

The location is predicted in three-dimensional space, such as providing Cartesian coordinates in a frame of reference of the surgical system or another frame of reference. Alternatively, the location is in two-dimensions, such as a location within an x-ray image.

The location may be for any lung object. For example, a location for an inter-segmental lymph node, mass, nodule, or landmark is predicted.

The processor 340 is configured to predict the location using artificial intelligence. In one embodiment, the artificial intelligence outputs the location in response to input of information. In another embodiment, the artificial intelligence outputs information used to then determine the location, such as outputting control instructions or change in robotic arm position to place the instrument 1159 closer or adjacent to the lung object. The processor 340 is configured to apply the artificial intelligence and use the output to then determine the location. The processor 340 may be configured to perform other processing, such as pre-processing (e.g., normalization or scaling) of the input to the artificial intelligence or post-processing of the output of the artificial intelligence (e.g., applying heuristics and/or guidance to place the instrument 1159 by the lung object).

The artificial intelligence outputs in response to an input. Any of various inputs may be used. For example, the input includes one or more pre-operative images of the patient. The images may be scan data representing the patient in one, two, or three dimensions. For example, the pre-operative image is a CT or magnetic resonance image representing a volume or slice of the patient. Other medical imaging, such as positron emission tomography, single photon emission tomography, x-ray, or ultrasound images may be used.

The input also includes a position of the lung object in the pre-operative image of the patient. The location in one, two, or three dimensions reflected in the pre-operative image is designated, such as through pre-operative planning. The location may be a point (e.g., center or spot on a surface of the object) or a region (e.g., segmentation of the object).

Another alternative or additional input is patient clinical data. For example, the age, sex, body mass index, smoking history, diagnosis, or other information for the patient is input. Any clinical data that correlates with pre- and intra-surgical object location may be input.

As another alternative or additional input, a real-time image is input. A current image or sequence of images acquired during the surgery (while the surgery occurs) is input. For example, a real-time x-ray or a CT image acquired during surgery is input.

In further embodiments, other inputs include a detected breathing cycle, breathing sensor signals, instrument location (e.g., electromagnetic field derived instrument location), heart cycle information, and/or patient pose.

The artificial intelligence generates one or more outputs. For example, the output is the location given in any coordinate system, such as relative to one or more landmarks of the patient. The location may be output as coordinates, an image showing the location, and/or a location on an image. In another example, the output is one or more regions for the object. The regions may be parameterized in any way, such as central location and radius, parameters defining a box, parameters defining a sphere, parameters defining an ovoid, or a mesh defining any shape of region. The machine-learned model 310 outputs the location or position of the lung object in response to the input of the information. The location or position is of the lung object while the surgery is occurring, such as the location of the lung object in a deflated or deformed lung as compared to the location of the lung object in the pre-operative image.

The artificial intelligence is a machine-learned model 310. In the example embodiment of FIG. 3, the machine-learned model 310 stored in the memory 300 is configured by training to receive the input (e.g., pre-operative image with object location and intra-operative image without location) and generate the output (e.g., the intra-operative location of the lung object).

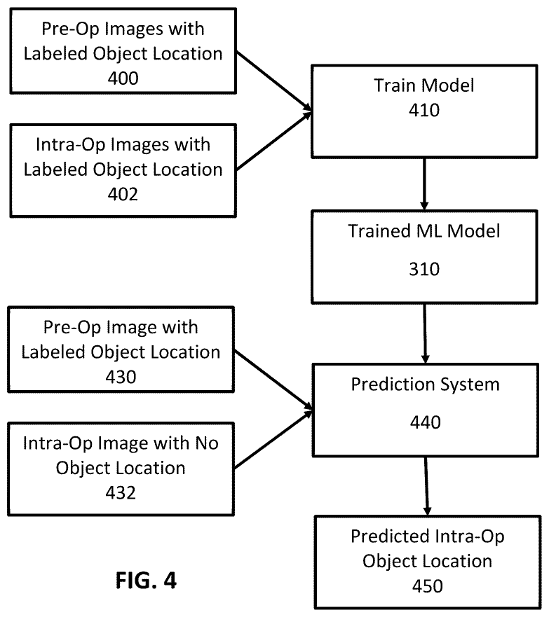

The machine-learned model 310 was previously trained using machine learning as represented in FIG. 4. Machine learning is an offline training phase 410 where the goal is to identify an optimal set of values of learnable parameters of the model 310 that can be applied to many different inputs (i.e., previously unseen inputs for a given patient). In this example, the training data is many samples of input pre-operative images with labeled mass locations 400 and intra-operative images with labeled mass location 402 as the ground truth. After training 410, a trained ML model 310 results. The trained ML model 310 includes the values of the machine-learned parameters of the model. The machine-learned parameters can subsequently be used during operation (testing or application phase) by the prediction system 440 to rapidly generate the output. Once learned, the machine-learned model 310 is used in an online processing phase in which the inputs (e.g., pre-operative image with labeled object location 450 and intra-operative image with no object location 432) are input, and the location 450 or information used to determine the location 450 is output based on the model values learned during the training phase. The location 450 of the lung object for the lung subjected to surgery (intra-operative location) is output.

Returning to FIG. 3, various types of machine learning models may be used, such as support vector machines, neural networks, Bayesian, regression, or another. In one embodiment, the model is a neural network, such as a fully connected neural network or a convolutional neural network. For example, a multi-layer perceptron neural network is used. As another example, a regression model, such as linear, logistic, ridge, Lasso, polynomial, or Bayesian linear regression model, is used. Any architecture or layer structure for machine learning may be used, such as U-Net, encoder-decoder 312, or image-to-image network. For example, a neural network with a plurality of different layers, types of layers, and/or activation functions is used. For the encoder-decoder 312, the encoder includes layers and downsampling creating features with greater abstraction, and the decoder includes layers and upsampling to generate the output as a spatial distribution. The architecture defines the structure, learnable parameters, and relationships between parameters.

The model 310 is machine trained. Deep or other machine training is performed. Many (e.g., hundreds or thousands) samples of inputs to the model and the ground truth outputs are collected as training data. For example, data from testing (e.g., on cadavers) or from usage (e.g., from surgeries performed on patients) is collected as training data. The inputs are obtained, and the resulting output is determined during the testing or surgery. Simulation may be used to generate the training data. Experts may curate the training data, such as assigning ground truth for collected samples of inputs. Training data may be created from a guideline or statistical shape model. Many samples for a given surgery may be collected.

The machine training optimizes the values of the learnable parameters. Backpropagation, RMSprop, ADAM, or another optimization is used in learning the values of the learnable parameters of the model 310. Where the training is supervised, the differences (e.g., L1, L2, or mean square error) between the estimated output (e.g., centroid location and radius) and the ground truth output are minimized.

Once trained, the model 310 is applied during MIS or other surgery for a given patient. For example, the machine-learned model 310 is used to determine the location of a lung object, which location is then used for surgery for the given patient by a given surgeon. The machine-learned model 310 is previously trained, and then used as trained. Fixed values of learned parameters are used for application. The learned values and network architecture determine the output from the input. During application for pre-operative planning for a patient, the same learned weights or values are used. The training sets or establishes the operation of the model 310 as trained. Different training, architecture, and/or training data may result in different prediction. The model and values for the learnable parameters are not changed from one patient to the next, at least over a time (e.g., weeks, months, or years) or number of surgeries (e.g., tens or hundreds). These fixed values and corresponding fixed model are applied sequentially and/or by different processors to inputs for different patients. The model may be updated, such as retrained, or replaced, but does not learn new values as part of application for a given patient.

The output interface 322 is a network card, graphics interface (e.g., HDMI), memory interface, or another hardware interface for communicating data from the processor 340 and/or memory 300 to another device. The output interface 322 is configured by hardware, software, and/or firmware to output the location of the lung object as predicated. The predicted location or locations are output by the output interface 322.

In one embodiment, the output interface 322 connects to the display 324. An image including the predicted location is output. The display 324 is a screen, printer, or another hardware device that displays the image. The image may be the coordinates of the location or representation of the patient with the location (e.g., CT with the location marked in the image). The display shows the predicted location.

In another embodiment, the output interface 322 outputs the location to a navigation system, such as a surgical robotic system. A controller 326 of the surgical system, a bronchoscope navigation system, and/or a surgical robotic system receives the location. The instrument is controlled and/or the location is displayed by the controller 326 based on the predicted location.

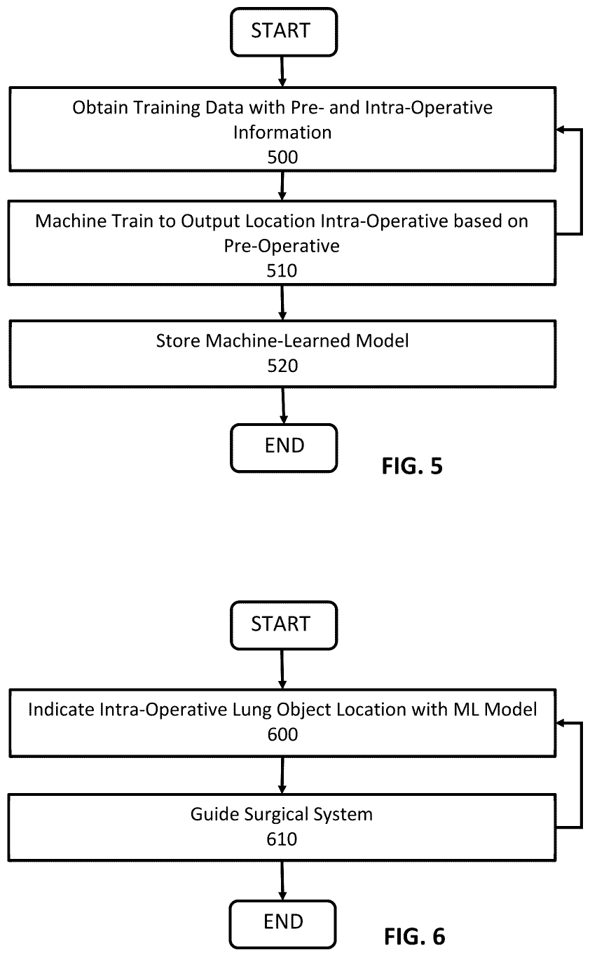

FIG. 4 illustrates a general framework for machine learning and application of the machine-learned model. FIG. 5 is a flow chart diagram of one embodiment of a method for machine training 410. FIG. 6 is a flow chart diagram of one embodiment of a method for application of the machine-learned model 310.

FIG. 5 is a flow chart diagram of one embodiment of a method for machine training for intra-operative lung object location predication. The acts are performed in the order shown (top-to-bottom or numerical) but may be performed in other orders. Additional, different, or fewer acts may be used. For example, an act for applying the machine-learned model 310 is provided without or in addition to the storing in act 520.

The system of FIG. 3 or a different system performs the acts. For example, a computer or server with access to a database storing the training data performs the machine training. The resulting trained model is stored in the database and/or a different memory.

In act 500, a processor obtains training data. The training data is obtained from a memory, such as a database, transfer over a computer network, and/or by loading from a removable storage medium. Alternatively, or additionally, the training data is obtained by mining or searching patient records or data stored from prior operations. The training data may be created by expert curation, simulation, experiments, studies, and/or automated extraction.

For machine training, hundreds, thousands, or more samples of input and outputs are obtained. For example, many samples of pre-operative images of lungs with designated or labeled lung objects and intra-operative locations of the lung objects with the lungs deflated relative to the lungs of the pre-operative images are acquired. Other information may be obtained, such as additional inputs. For example, intra-operative images are provided for some or all the samples. The images are CT, but different modalities may be used for all or for some of the images.

Other information may be included in the samples. For example, patient clinical data, breathing cycle, heart cycle, and/or patient pose information is obtained.

The locations for the pre-operative images and/or the ground truth are locations with reference to a common frame of reference, such as the patient and/or surgical system. For example, Cartesian coordinates or other parameterization with reference to the surgical system are used. As another example, vectors or other parameterization with reference to one or more patient landmarks (e.g., anatomical marker or fiducial) are used.

The images may be pre-processed. For example, registration is used to align the images. Normalization may be used to normalize the intensities of the images. Scaling or other transformation may be used to alter one or more images to a same standard.

The machine learning model has defined inputs and outputs. Each of the training samples obtained include sets of the inputs and corresponding output.

In act 510, the processor machine trains the machine learning model. The processor trains the model to generate the output location or locations of the intra-operative lung objects in response to input of the information (e.g., pre-operative image with lung object location labeled, clinical data, and/or intra-operative image). Using the many samples in the training data, the machine training optimizes the values of the learnable parameters of the defined model architecture to generate the output object locations.

In act 520, the processor stores the machine-learned model in a memory. The architecture and the values of the learned (learnable) parameters are stored. The model as learned is stored for application by the same processor or a different processor. The machine learning model as trained is stored for use with a patient (i.e., with unseen inputs) to generate an intra-operative location of a lung object.

FIG. 6 shows one embodiment of a method for intraoperative guidance to a lung object for a surgical system (e.g., 1000, 1136). A machine-learned model 310, as trained, is used to output a location of a lung object. A mass, a nodule, an intra-segmental lymph node, and/or a landmark are output for assistance in guiding surgery.

The method of FIG. 6 is performed by the surgical system of FIG. 1, 2, or 3, another surgical robotic system, or a surgical system without robotics. In one embodiment, the surgical robotic system of FIG. 3 performs the method. An interface receives the images of a patient (e.g., pre-operative image and intra-operative image) and location of a lung object at the pre-operative stage. The interface receives the input from user input, transfer over a computer network, and/or loading from memory. The programmed processor 340 (also referred to here as processing logic or circuit) performs the input to the artificial intelligence and generates the output indication in act 600 using the artificial intelligence. The processor guides the surgical system in act 610 based on the output indication of location of the lung object during surgery. A display 324 may display the indication and/or a controller 326 may use the indication for robotic control. A non-transitory memory 300 may store instructions for the programmed processor 340 to determine the location(s) and/or the machine-learned model or models 310.

The acts are performed in the order shown or a different order. Additional, different, or fewer acts may be provided. For example, acts for performing surgery are included. The return arrow from act 610 to act 600 represents repetition in an on-going or periodic manner during the surgery.

In act 600, a processor 340 indicates a location of a lung object during surgery. The intra-operative location of the object is indicated to assist in surgery. Since the lung deforms during surgery, such as deflates, the pre-operative location of the same object may be different than the intra-operative location. The processor 340 indicates the location during the surgery to assist in the surgery.

The processor 340 generates the indication of the location in response to input of information to a machine-learned model 310. Information is input to the machine-learned model 310. Various information may be gathered, such as pre-operative and intra-operative images as well as an indication of a location of a lung object relative to the pre-operative image. The intra-operative image may be a real-time image, such as an image acquired by scanning during or in the surgery. The images may be of any modality, such as CT, camera (e.g., bronchoscope), and/or x-ray (e.g., pre-operative image is a CT image with an annotation or label for the location of the lung object and an intra-operative (real-time) x-ray, camera, or CT).

In response, the machine-learned model 310 generates the output indication. The processor 340 indicates the location as an output of coordinates, graphic overlay on an image, and/or an image including the object. Other output indications may be used. The machine-learned model 310 outputs one or more locations of a respective one or more objects or parts of or in the lungs of the patient. The location of the object is provided for the deflated or deformed lung during surgery. The intra-operative location of the object designated on the pre-operative image or planning is predicted.

Any machine-learned model may be used. For example, a neural network, such as a fully connected or convolutional neural network, is used. In one embodiment, the model receives one or more images and outputs one or more images, such as U-Net, encoder-decoder, or another image-to-image network. The output image shows a representation of the lungs and a location of the lung object or shows the location of the lung object as a spatial representation in two or three dimensions. In another embodiment, the model is a neural network, support vector machine, regressor, or other model to output coordinates or vectors for any given frame of reference. The coordinates or vectors indicate the location of the object.

In one embodiment, breathing information is input to the machine-learned model 310. Measurements from a breathing sensor and/or indication of a part of the breathing cycle for the pre-operative image and/or the intra-operative image are input. The machine-learned model 310 may have been trained to account for breathing in location estimation as the movement of the diaphragm and resulting movement of the lungs may alter the location of the lung object. Additionally, or alternatively, clinical data may be input for generating the output indication. The clinical data (e.g., body mass index and/or age) may correlate with amount of deformation of the lungs or other location-deterministic effect.

In an additional embodiment, the same machine-learned model 310 or another machine-learned model outputs an image of the lung during surgery. For example, a trained image-to-image network outputs a segmentation of the lungs to show the deformation and a location of a lung object (e.g., lung mass).

In act 610, the surgeon and/or controller 326 guide the surgical system to the location based on the indication. The indication of the location provides a visual and/or spatial guide so that the bronchoscope or other instrument can be positioned within the patient relative to the lung object. For example, the location is used to guide the bronchoscope to adjacent the lung object for viewing the lung object. Since the location is in or on the lungs during surgery, the guiding accounts for the deformation of the lungs due to surgery. As a result, the instrument 1159 may be more quickly, efficiently, and/or accurately guided to the lung object.

The surgeon may manually operate the surgical system. For example, the surgeon directly controls the bronchoscope, such as by steering wires and/or pushing and pulling. By viewing an image showing the location, the surgeon may directly control the bronchoscope to guide the bronchoscope to the location. As another example, the surgeon controls robotics or other motor, which then moves the bronchoscope. By viewing an image showing the location, the surgeon may control the bronchoscope through robotics to guide the bronchoscope. In yet another example, the controller 326 executes instructions to control the robotics and guide the bronchoscope to the location without user control in guiding.

FIGS. 7-9 show examples of different approaches or use cases for guiding during surgery. Other use cases may occur.

In FIG. 7, the predicted location 450 of the object and a real-time image 700, such as from a camera, the bronchoscope, or medical scanner, are provided to an image processing system 710, such as the controller or another processor. The image processing system 710 creates an image overlay 720 of the location as a graphic on the real-time image 700 generated during the surgery. The process repeats during the surgery. The images 700 with the overlays 720 are displayed, enabling the surgeon to manually or robotically guide the tool 1159 to an appropriate location.

The processor 340 generates an indication of the object location. The indication is displayed by the image processing system 710. In one embodiment, the indication is a graphic overlay on the pre-operative image 430 and/or the real-time image 700, such as showing a camera image of the patient with a graphic having solid lines for directly viewable or dashed lines where tissue is between the bronchoscope and the lung object. Other graphics to assist in guiding may be provided, such as line showing a path to follow to the predicated location 450 of the lung object.

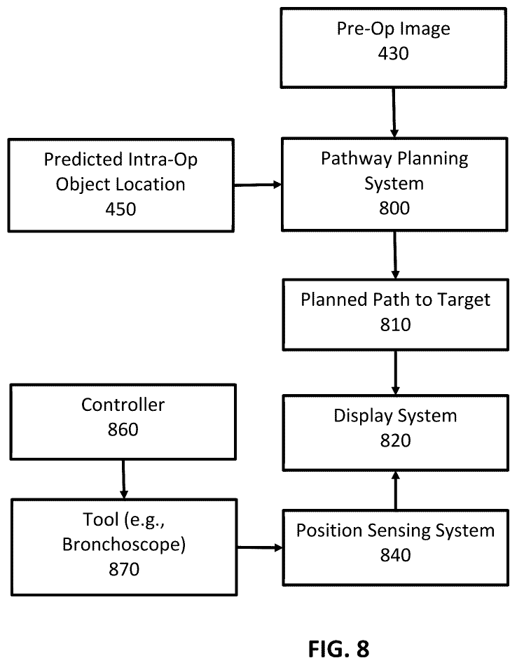

In FIG. 8, the location of the surgical instrument 1159 is detected and used with the location 450 of the lung object to guide the instrument 1159 in the surgical system. The pre-operative image 430 and the lung object location 450 are provided to a pathway planning system 800, which generates a planned path 810 to follow for the instrument 1159 to reach the lung object. The display system 820 displays an image of the planned path 810. A position sensing system 840 detects a position of the surgical tool 870. Electric field or electromagnetic sensing may be used to detect the position of the tool 870. Alternatively, medical scanning, such as CT or x-ray, may be used to detect the position of the tool 870. The location of the tool 870 is displayed on the display system 820. The surgeon then controls the tool 870 with the controller 860 to guide the tool 870 to the lung object along the planned path. The locations of the lung object and the detected tool 870 are used to guide the tool 870 to the lung object.

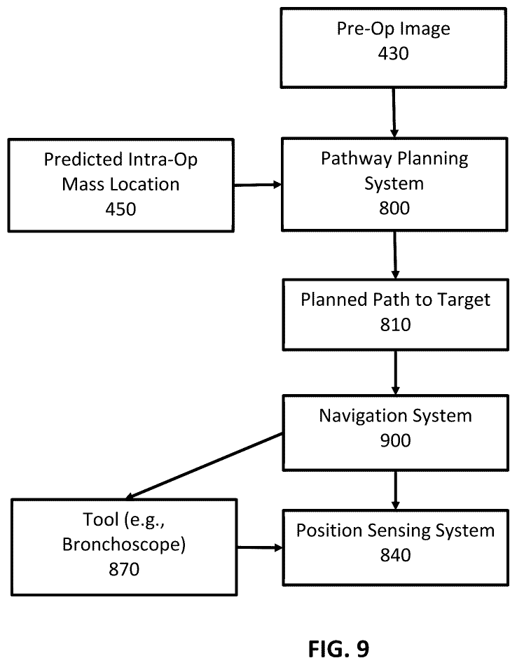

FIG. 9 shows an alternative to FIG. 8. The controller 860 guides the tool 870 without steering or translation input by the surgeon in the surgical system. In one embodiment, the navigation system 900 is part of a surgical robotic system that controls a robot to steer and/or translate the tool 870. For example, a surgical robotic arm is controlled by the navigation system 900 to guide the surgical tool 870 to the location 450 of the lung object. Rather than or in addition to a display system 820 to display the locations for surgeon-based guidance, the locations are provided to the navigation system 900. The navigation system 900 includes a controller to control the tool 870 for automated guidance of the tool 870 to the lung object location 450. The locations of the tool 870 as detected by the position sensing system 840 and the predicted lung object are used to guide the tool 870 to the lung object along the planned path 810.

In one embodiment, the surgical robotic system has a coordinate system aligned with the patient, position sensing system, pathway planning system 800, navigation system 900, and/or pre-operative image 430. The surgical robotic system then moves the surgical instrument 870 to the location automatically.

The location of the lung object may be output to memory, a display, robotic system controller, medical record, and/or a report. The processor 340 generates the indication of the location as text, graphic, or image. For example, the user interface displays the indication. The display is configured by loading an image with the indication into a display buffer or plane of the display device.

In one embodiment, a method is provided for intra-operatively predicting a location of a lung mass in a deflated lung. A machine learning model trained from a set of training images of pre-operative lungs and labels indicating observed locations of lung masses in the pre-operative lungs and observed locations of the lung masses in corresponding deflated lungs is obtained. A pre-operative image of a target lung is obtained. A pre-operative location of a target lung mass in the pre-operative image of the target lung is obtained. The machine learning model is applied to the pre-operative image of the target lung, with or without a real-time or surgical image of the target lung, to predict an intra-operative location of the lung object (e.g., mass) based on the pre-operative location of the lung mass. The intra-operative location is for when the lung is in a deflated state. An output indicative of the predicted intra-operative location is generated.

In further embodiments, a visualization of the predicted intra-operative location is shown; the predicted intra-operative location is provided to a bronchoscope navigation system for guiding a surgical tool to the predicted intra-operative location; and/or a surgical robot arm is controlled to guide a surgical tool to the predicted intra-operative location.

The above description of illustrated embodiments of the invention, including what is described below in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize. These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A method for intraoperative guidance to an object in a patient for a surgical system, the method comprising:
indicating a location of the object during surgery, the location indicated by a machine-learned model, the machine-learned model outputting the location in response to input of information to the machine-learned model; and
guiding the surgical system to the location based on the indication.

2. The method of claim 1 wherein guiding comprises overlaying the location on an image generated during the surgery.

3. The method of claim 1 wherein guiding comprises guiding based on detected location of a surgical instrument relative to the location of the object.

4. The method of claim 3 wherein guiding comprises guiding by a controller of the surgical system using the detected location of the surgical instrument and the location of the object.

5. The method of claim 1 wherein indicating comprises indicating by the machine-learned model, the machine-learned model comprising a convolutional neural network, a U-Net, or an encoder-decoder.

6. The method of claim 1 wherein indicating comprises indicating the location by the machine-learned model in response to the information, the information comprising a pre-operative image of a patient and a real-time image of the patient in the surgery.

7. The method of claim 6 wherein the pre-operative image comprises a computed tomography image and the real-time image comprises a computed tomography or x-ray image.

8. The method of claim 1 wherein indicating comprises indicating where the information includes a pre-operative image of a lung and where the location is on the lung with the lung deflated relative to the lung in the pre-operative image, the location being a predicted intra-operative location.

9. The method of claim 1 wherein the object comprises a mass, a nodule, or an intra-segmental lymph node.

10. The method of claim 1 wherein guiding comprises guiding, in a patient, a bronchoscope of the surgical system.

11. The method of claim 1 wherein guiding comprises controlling a surgical robotic arm to guide a surgical instrument to the location.

12. The method of claim 1 wherein indicating comprises indicating with the information comprising patient clinical data and/or breathing cycle data.

13. A medical system for intra-operative prediction of a location of an object in a patient, the medical system comprising:
a memory configured to store a machine-trained model, the machine-trained model having been trained from training images comprising pre-operative images of sample objects and ground truth comprising positions of the sample objects during surgery;
a processor configured to predict the location of the object for a patient during the surgery, the location predicted by the machine-trained model in response to input of a pre-operative image of the patient and a position of the object in the pre-operative image to the machine-trained model; and
an output interface configured to output the location of the object as predicated.

14. The medical system of claim 13 wherein the output interface connects to a display, the location shown on the display.

15. The medical system of claim 13 wherein the output interface is configured to output the location to a bronchoscope navigation system or a surgical robotic system.

16. The medical system of claim 13 wherein the input to the machine-trained model comprises the pre-operative image of the patient, the position of the object in the pre-operative image, and patient clinical data.

17. The medical system of claim 13 wherein the pre-operative image of the patient comprises a computed tomography image, and wherein the input further includes a real-time x-ray or computed tomography image of the patient during the surgery.

18. A method for machine training for intra-operative object predication, the method comprising:
obtaining pre-operative images of lungs with objects and intra-operative locations of the objects with the lungs deflated relative to the lungs of the pre-operative images;
machine training a machine learning model to output the locations of the intra-operative objects in response to input of the pre-operative images; and
storing the machine learning model as trained.

19. The method of claim 18 wherein machine training comprises machine training for the output in response to input of the pre-operative images, pre-operative locations of the objects, intra-operative images of the lungs, and clinical data.

20. The method of claim 19 wherein obtaining comprises obtaining the pre-operative images and the intra-operative images as computed tomography images.
